(19) United States
(12) Patent Application Publication
(10) Pub. No.: US 2006/0263855 A1
(43) Pub. Date: Nov. 23, 2006

(54) Intein-mediated protein purification using in vivo expression of an elastin-like protein

(76) Inventors: David W. Wood, Princeton, NJ (US); Mahmoud R. Banki, Princeton, NJ (US)

Correspondence Address: CONNOLLY BOVE LODGE & HUTZ, LLP, P O BOX 2207, WILMINGTON, DE, 19899, US

(21) Appl. No.: 11/374,403

(22) Filed: Mar. 13, 2006

Related U.S. Application Data
(60) Provisional application No. 60/661,559, filed on Mar. 14, 2005.
(60) Provisional application No. 60/703,185, filed on Jul. 28, 2005.

(51) Int. Cl. C07K 14/78; C07H 21/04; C12P 21/04; C12N 1/21; C12N 15/74

(52) U.S. Cl. 435/69.7; 435/252.33; 435/488; 530/350; 536/23.5

(57) ABSTRACT

Purification of recombinant proteins is performed by expressing in a host cell a fusion protein comprising: (a) a product protein domain, (b) an intein, and (c) at least one aggregator protein domain, wherein the aggregator protein domain comprises a self-aggregating protein such as elastin-like proteins (ELPs).

### CROSS REFERENCE TO RELATED APPLICATIONS

This application asserts priority to U.S. Provisional Application Nos. 60/661,559 filed Mar. 14, 2005; and 60/703,185 filed Jul. 28, 2005, each of which is incorporated herein by reference in its entirety.

### GOVERNMENT LICENSE RIGHTS

The U.S. Government may have certain rights in this invention as provided for by the terms of grant W911NF-04-1-0056 awarded by the Army Research Office.

### FIELD OF THE INVENTION

The invention is directed generally to methods and compositions for purification of recombinant proteins. More particularly the invention is directed to a method for bioseparation using a fusion protein comprising the desired protein, a self-cleaving intein, and an elastin-like protein (ELP). The fusion protein is reversibly non-soluble. The non-soluble components including the fusion protein are then separated from the soluble components of the cell culture system and optionally washed. The fusion protein may be further purified by being rendered soluble. The fusion protein is then cleaved by activating the self-cleaving intein. This releases the desired product protein into solution where it can be recovered independent of the intein and ELP domain.

In a preferred method of the invention, the host cell produces the desired fusion protein.

### BACKGROUND OF THE INVENTION

Advances in protein expression systems have made possible the production of virtually any oligo peptide or polypeptide product. After expression, however, these products must often be purified for further use. Thus the rapid and economical purification of recombinant proteins represents a persistent challenge in the field of biotechnology. Protein purification typically involves several chromatographic steps, each optimized for each product protein. Each step can be costly and time-consuming, and inevitably decreases the final yield of the product. In the large-scale manufacture of recombinant proteins for industrial and therapeutic use, downstream purification is very costly and can account for up to 80% of the total production cost. The development of simple and reliable methods for protein purification, which can be applied to many products at laboratory to manufacturing scales, is therefore an important goal in bioseparations technology development.

The purification of protein may be obtained by the addition of an affinity tag nucleic acid sequence to a nucleic acid sequence which encodes a target protein. LaVillie et al., Biotechnology 6:501-506 (1995). This process results in the expression of an affinity-tagged target protein that can be purified by exploiting the highly selective binding characteristics of the tag. Once the affinity-tagged target protein is purified, the tag can be enzymatically removed by hydrolysis with an appropriate protease enzyme. Recovery of a native target protein, which is often necessary for many applications, requires the proteolytic removal of the affinity tag. The potential of this technique for use in large scale production is limited in part by complications arising from the addition of protease to the purified fusion protein solution. The protease may cause nonspecific cleavage within the target protein, leading to the destruction of the target protein. A second disadvantage is cost, as protease is expensive. Particularly for industrial applications, protease cost may be a determining factor in selecting a separation system. Also, the addition of protease necessitates an additional purification step for protease removal, which increases costs.

Another method for protein purification involves the creation of a fusion protein in which an intein is inserted between the desired product protein and an affinity binding protein, effectively generating a self-cleaving tag. Discovered in 1990, inteins are naturally occurring internal interruptions in a variety of host proteins. Hirata et al., J. Biol. Chem. 265:6726-6733 (1990); Kane et al., Science 250:651-657 (1990); Perler et al., Nucl. Acids Res. 22:1125-1127 (1994); and Noren et al., Angew. Chem. Int. Ed. 39:450-466 (2000). Inteins are a widely-distributed class of self-splicing protein elements. Protein splicing is a form of posttranslational processing that involves the excision of an intervening protein sequence from a host protein. Concomitantly the flanking polypeptides are joined. The intervening protein sequence is known as an intein, while the flanking sequences are called exteins.

Structural analysis suggests that inteins are generally composed of an endonuclease protein domain and a self-splicing mini-intein domain. The endonuclease domain is not necessary for splicing. Indeed, the endonuclease domain can be deleted to yield a functional splicing mini-intein. One example of a mini-intein is the deletion of the entire endonuclease component from the Mycobacterium tuberculosis recA gene, which reduces the 440 amino acid intein to a functional mini-intein of 168 amino acids.

The genetic elements that encode inteins must be in-frame insertions in a gene with the mature protein product being the same size as the homologs lacking the intein insertion. In addition, the presence of specific splice junctions is necessary. The requisite splice junctions for inteins are serine (Ser, S), threonine (Thr, T) or cysteine (Cys, C) at the intein N-terminus and the dipeptide histadine-asparagine (His-Asn, H-N) or histidine-glutamine (His-Gln, H-Q) at the C-terminus. Ser, Thr, Cys and Asn are necessary residues in the splicing mechanism, and act as nucleophiles to create an N—S or N—O acyl rearrangement, depending on the residue. This forms a linear thioester or ester intermediate. Extein ligation follows, mediated by the highly conserved cysteine, serine or threonine immediately following the intein. Acting as a nucleophile, the sidechain of this residue attacks the ester bond formed in the first step, resulting in transesterification. A branched intermediate is formed. Next, the intein is released when the asparagines at the end of the intein cyclize to form a succinimide. Lastly, an O—N or S—N acyl rearrangement converts the ester linking the exteins to a peptide bond.

Intein function can be modified. For example, a modified intein cleaves instead of splices. Specifically, when an inteins' N-terminal Cys is replaced with an Ala, N-terminal cleaving and splicing is eliminated with C-terminal cleavage observed. Replacing the Asn in the C-terminal with Ala stops C-terminal cleavage and splicing and results in N-terminal cleavage. Other conditions result in cleavages at both the N— and C-terminals, in place of splicing. In the case of C-terminus cleaving, the requirement for a cysteine, serine or threonine immediately following the intein is eliminated.

Thus blocking certain splicing steps permitted the development of self-cleaving affinity tags. Wood and co-workers used the Mycobacterium tuberculosis (Mtu) RecA intein for protein purification with C-terminal cleavage of the target protein. Biotechnol Prog 16(6): 1055-63 (2000). Wood and colleagues also characterized Mtu RecA inteins with the endonuclease domain deleted, creating mini inteins. Furthermore, they were able to create mutated rapid-splicing and cleaving varieties. Characterization showed that the mini-cleaving intein ΔI-CM was very useful for protein purification. Wood et al., Nature Biotechnol. 17(9):889-92 (1999).

Chong and colleagues developed a single-column purification system using the vacuolar ATPase intein subunit of Saccharomyces cerevisiae (Sce VMA intein). Nucleic Acids Res 26(22): 5109-15 (1998). In each case, the intein was inserted in between the affinity binding protein and the product gene. Cells were induced to overexpress precursor protein followed by conventional purification with affinity binding domains. In both cases, the product protein can then be cleaved from the intein affinity tag while on the column, allowing the recovery of the product protein without addition of protease. With the Mtu intein system, the intein cleaving is induced by shifting pH and temperatures. With the Sce intein system, intein cleaving is induced by mass action by the addition of thiol-containing compounds. Additional systems have now been reported that use similar strategies to both systems for inducing intein cleaving. Southworth et al., Biotechniques 27:110-20 (1999).

A remaining practical limitation to the use of self-cleaving affinity tags is the high cost of the affinity resins that are typically used in these separations. Also, the affinity resins often used with inteins have low binding capacity for the tagged fusion proteins, resulting in yield loss.

One method of protein separation that avoids affinity chromatography involves use of elastin-like polypeptides (ELPs). ELPs are capable of self-aggregation and precipitation. ELPs can be designed to spontaneously and reversibly fall out of solution above a phase transition temperature (Tₜ). Tₜ is most sensitive to the composition and molecular weight of the ELP, but can also be affected by salt concentration. U.S. Pat. No. 6,852,834 (Chilkoti) discloses fusion proteins consisting of a desired product protein and an ELP domain. Thermally stimulated phase-transition of the fusion protein is observed, which is used for protein separation.

### SUMMARY OF THE INVENTION

The invention is directed generally to a rapid and highly effective method for preparing substantially purified recombinant protein. The method is highly scaleable and relatively inexpensive. The invention is also directed to fusion proteins, plasmids, cells and compositions useful in the method.

The invention avoids the disadvantages of prior art affinity purification because no proteases need be used. Furthermore, the present technology avoids harsh chemical environments. The present invention further eliminates the requirement for conventional affinity tags as well as associated resins and apparatus. The present technology is useful for the expression and extraction of a wide range of proteins. The present invention will permit high quality, low cost preparations of isolated and purified proteins for laboratory and industrial use, such as for purification of industrial enzymes, veterinary products and pharmaceutical products.

In one aspect the invention is directed to a fusion protein comprising a product protein domain, a self-cleaving intein, and at least one aggregator protein domain, wherein the aggregator protein domain is capable of self-association and precipitation and comprises one or more elastin-like protein (ELP) domains. The intein is located between the product protein domain and the aggregator protein domain. The aggregator protein domain may be one or more ELP domains. The ELP domain preferably comprises the sequence (Val-Pro-Gly-Xaa-Gly)ₙ, (SEQ ID NO:1) wherein n has a value from one to about 480 and Xaa is the same or different and is any natural or synthetic amino acid. SEQ ID NO:1 with n=1 is the monomer unit of ELP. More preferably, Xaa is the same or different and is Val, Ala, or Gly.

In one embodiment, the product protein domain, the intein, and the aggregator protein domain are encoded by a single open reading frame in a nucleotide. In another embodiment, a linker peptide is linked to at least one aggregator protein domain.

The invention also is directed to nucleic acids encoding the fusion proteins of the invention, plasmids comprising the nucleic acids, cells stably transfected with the nucleic acids, and methods of producing the fusion proteins by culturing the cells.

The invention also concerns methods of purifying a product protein from a recombinant cell culture comprising:
(a) expressing a nucleotide encoding the fusion protein comprising an ELP aggregator protein domain in a host cell;
(b) allowing the fusion protein to leave the host cell by cell secretion or as a result of cell lysis;
(c) removing insoluble cell culture components from the cell culture to form a first suspension containing the fusion protein;
(d) adjusting one or more of the conditions of temperature, salt content, pH and solvent content of the first suspension to cause self-aggregation and precipitation of the fusion protein forming a first precipitate;
(e) separating the unprecipitated components from the first precipitate;
(f) adding water or solvent to the first precipitate and adjusting one or more of the conditions of temperature, salt content, pH and solvent content such that the fusion protein is resuspended to form a second suspension;
(g) adjusting one or more of the conditions of temperature, salt content, pH and sulfhydryl level of the second suspension such that the intein self-cleaves from the product protein to form an ELP-intein fusion and a separated product protein;
(h) adjusting one or more conditions of temperature, salt content, pH and solvent content such that the ELP-intein fusion self-aggregates and precipitates while the separated product protein remains in solution; and
(i) separating the solution of separated product protein from the ELP-fusion precipitate to yield a substantially purified product protein.

The invention also comprises the protein product isolated by the method of the invention.

### BRIEF DESCRIPTION OF THE FIGURES

FIG. 1 illustrates two schemes for protein purification using, on the left, an intein sequence and an ELP-domain and, on the right, affinity chromatography and an added protease.

FIG. 2 illustrates a general scheme of generating ELP tags of various lengths, and their attachment to an intein and product (target) protein.

FIG. 3 shows the aggregation states of an ELP fusion protein relative to the ELP transition temperature (Tₜ). Tₜ is the phase transition temperature of the fusion protein.

FIG. 4 illustrates a general scheme for purification of a product protein by intermediate production of an ELP-fusion protein.

FIG. 5 shows purification of chloramphenicol acetyl transferase (CAT) as identified by SDS-PAGE.

FIG. 6 illustrates SDS-PAGE of the purification of (a) α-hemoglobin stabilizing protein (AHSP), (b) β-lactamase (β-lac), (c) β-galactosidase, (d) E. coli catalase (katG), (e) green fluorescent protein (GFP), (f) glutathione S-transferase (GST), (g) maltose binding protein (M), (h) Nus A, (i) experimental protein 1 (EX1), (j) experimental protein 2 (EX2), (k) S824 and (l) experimental protein 3 (EX3).

FIG. 7 illustrates SDS-PAGE showing the full dissolution of AHSP fusion protein, β-lactamase fusion protein, CAT fusion protein, and glutathione-S-transferase fusion protein, thus indicating high level recovery of fusion proteins following purification.

FIG. 8 is a vector map of pUC-18-ELP₇₀.

FIG. 9 illustrates SDS-PAGE of the purification of GFP using crossflow filtration.

### DETAILED DESCRIPTION OF THE INVENTION

The present invention is envisioned to be used to purify any full size protein, polypeptide or oligo-peptide. As used herein “protein” and “polypeptide” are synonymous. More specifically, the product proteins include, but are not limited to, regulatory factors such as hormones and cytokines; therapeutic polypeptides such as blood products (including coagulation factors), vaccines, and growth hormones; enzymes useful for industrial application such as proteases; remediation enzymes such as organo phosphohydrolases; polynucleotide restriction enzymes; starch hydrolases for mono- and oligo-saccharide manufacture; and antibodies for diagnostic and therapeutic applications. Further, the system can be used in high through-put screening for the parallel purification of large libraries for research purposes. These uses can include proteomic studies as well as directed evolution and novel enzyme identification studies.

The fusion proteins of the present invention are proteins encoded by multiple in-frame nucleic acid sequences each directed to different protein domains or other copies of the same protein.

In the invention, an intein is used between the product protein domain and the aggregator protein domain as a readily cleavable element that can be used to release the product protein from the fusion protein after purification steps are performed. The inteins used in the present invention are self-cleaving elements in which cleavage can be controlled by pH, temperature, salt concentration, free sulfhydryl concentration and other means that do not involve contact of the intein with an external protease. Intein self-cleavage can be induced by a trigger specific to the intein. Common triggers include addition of a reducing agent such as a thiol and a decrease in pH, for example, from pH 8.5 to 6.0. In one embodiment of the invention, the fusion protein has an intein bound to the product protein at the C-terminus of the intein. In such a fusion protein, intein self-cleavage is desired at the C-terminus, which can be accomplished by change in pH and/or temperature typically. In another embodiment of the invention, the fusion protein has the intein bound to the product protein at the N-terminus of the intein. In this instance, intein self-cleavage is desired at the N-terminus, which may be accomplished by altering the free sulfhydryl concentration.

Preferred are so-called “mini-inteins” in which the endonuclease domain has been deleted, rendering the intein smaller yet still capable of self-cleavage. Examples of such inteins are the pH-sensitive mutant inteins described in Wood et al. Nature Biotechnology 17: 889-892 (1999). Particularly useful is ΔI-CM intein disclosed therein. The ΔI-CM intein is encoded by the sequence found at SEQ ID NO:2. A key feature of the ΔI-CM mutant is its extreme pH sensitivity, which allows purification of intact precursor followed by rapid C-terminal cleavage. Other useful inteins are found in U.S. Pat. No. 6,933,362 (Belfort et al.). An example of a useful intein is an intein derived from Mycobacterium tuberculosis (Mtu) recA intein that has only the first 110 amino acids and the last 58 amino acids of that 441-amino acid protein and mutants derived therefrom using methods known in the art. Such an intein is a truncated Mtu recA intein with the endonuclease domain deleted.

Preferred inteins for the present invention display rapid cleavage isolated at either the C-terminal or the N-terminal, more preferably at the C-terminal, and are highly controllable. The cleavage preferably is completed (about 90-95%) in four hours or less at 37° C. or longer at lower temperatures, which allows for easy scale-up. In one embodiment, the inteins used in the invention display a strong dependence on temperature, allowing uncleaved precursor to be expressed in host cells for purification as long as the culture temperature is below the cleavage temperature of the intein. Preferably, the self-cleaving intein yields optimized controllable cleavage rather than splicing. Furthermore, the intein should be as small as possible for this strategy to be attractive for scale-up. Preferred inteins exhibit a 20- to 40-fold increase in activity between pH 8.5 and 6.0. These pH values are relatively mild, decreasing the potential for damage to the product protein due to pH-induced denaturation, and thus allowing the recovery of pure protein with minimal damage. This small pH change also decreases the possibility that the binding domain will lose affinity during cleavage.

Preferably, the intein used allows for self-cleavage that releases the product protein in its native form. An example of such an intein is the C-terminal cleaving ΔI-CM. Other fusion proteins may be used in which self-cleavage of the intein results in modifications to the product protein requiring additional processing to obtain the product protein in native form. For example, in the configuration where the product protein is released by N-terminal cleavage, the cleavage reaction may require the addition of thiol containing compounds that modify the C-terminus of the product protein. Native protein is recovered only after subsequent hydrolysis of the cleavage-inducing reagent. Chong et al., J. Biol. Chem. 272:15587-15590 (1997).

Most preferred inteins are mini-inteins that display rapid, isolated C-terminal cleavage and are pH-sensitive. Such inteins obviate the need for reducing reagents and additional purification steps required for other inteins, such as the N-terminal cleaving inteins discussed supra, and have advantageous size and stability characteristics.

Useful inteins for the present invention include those that have a C-terminal histidine-asparagine. The fusion protein of the invention includes a product protein and an intein, wherein the C-terminal histidine or asparagine or histidine-asparagine of the intein is immediately followed by the second amino acid of the desired product protein. The second amino acid of the desired product protein can be lysine. The presence of the penultimate C-terminal histidine residue may confer pH sensitivity. Thus, it may be advantageous that the C-terminal penultimate histidine be present. Preferably the C-terminal asparagine is present for cleavage activity. More particularly, without necessarily wishing to be bound by any one particular theory, it is believed that the mechanism of intein cleavage requires that the final residue of the intein be asparagine (not histidine). The C-terminal histidine referred to herein can be the highly conserved histidine that immediately precedes the final asparagine. If the C-terminal histidine of the intein is immediately followed by the desired product protein and there is no asparagine residue at the final intein residue, then cleavage may not always be possible. The mention herein of a dipeptide at the end of the intein sequence can be interpreted as “Z-asparagine,” to show that the final asparagine residue of the intein is advantageously present for any cleavage, while the histidine residue that precedes it is thought to be responsible for the pH sensitivity of the intein, i.e., “Z” can be histidine. However, “Z” can be any suitable amino acid, such as an amino acid that confers pH sensitivity, e.g., pH sensitivity outside of the range of when “Z” is histidine; for instance, to shift the range of pH sensitivity of the intein.

In the present invention, the aggregator protein domain provides a protein region that is capable of associating with an ELP to form a complex having low solubility. Preferably, the aggregator protein domain is an ELP domain that self-aggregates or aggregates with the ELP domain of other fusion proteins. In this manner, the aggregator protein domain provides a mechanism to separate the fusion protein from the cell lysate or cell culture medium by phase. Chromatography is not required for purification, although it is envisioned that when very high purity is required, the purification method of the present invention may be followed by additional downstream purification steps.

The fusion protein can be separated from the cell lysate by centrifugation and separation of the supernatant, by diafiltration using ultrafiltration membranes, by flocculation, gas bubbling, or other methods known to those skilled in the art for separating solid and liquid phases.

The aggregator protein domain is an ELP domain or a plurality of the same or different ELP domains. The ELP domains are self-aggregating at their phase transition temperature (Tₜ). Thus, when the conditions of the solution are altered such that the phase transition temperature of the ELP fusion protein is reached, the fusion protein is precipitated by the aggregation of the ELP domains of the fusion proteins. Sufficient ELP domains must be present such that the fusion protein of the invention has an inverse phase transition. The phase transition of the fusion protein is preferably mediated by one or more means selected from the group consisting of changing temperature; changing pH; addition of organic solutes and/or solvents; salt addition; side-chain ionization or chemical modification; and changing pressure.

The preferred means for mediating the phase transition is raising temperature. Preferably, the fusion protein has a reasonable transition temperature above which the fusion protein collapses out of solution and the ELP domains self-aggregate. The transition temperature can be modulated by salt addition, where a higher salt generally leads to a decrease in transition temperature. A reasonable transition temperature is between 0° C. and 50° C., and preferably between 20° C. and about 40° C. Preferred ELPs are those that provide the fusion protein with a transition temperature (Tₜ) that is within a range that is not damaging to the target protein. Such suitable ELP domains may have molecular weights of at least 9,000 Daltons, and preferably have a molecular weight in the range of 10,000 to about 100,000 Daltons.

In another aspect of the invention the ELP domain comprises the sequence (Val-Pro-Gly-Xaa-Gly)ₙ (SEQ ID NO:1)ₙ, wherein n has a value from one to about 480 and Xaa is the same or different and is any natural or synthetic amino acid. In one aspect of the invention, Xaa is a natural amino acid. In one aspect of the invention, n has a value from ten to about 220. In a particular composition, n is about 110. In one aspect of the composition, Xaa is the same or different and is selected from Val, Ala, and Gly.

The ELP domain need not consist of only Val-Pro-Gly-Xaa-Gly to exhibit the desired phase transition. The oligomeric repeats may be separated by one or more amino acid residues that do not eliminate the phase transition of the fusion protein required for the present invention. In a preferred aspect of the invention, the ratio of Val-Pro-Gly-Xaa-Gly oligomeric repeats to other amino acid residues of the ELP domain is greater than about 75%, more preferably, the ratio is greater than about 85%, still more preferably, the ratio is greater than about 95%, and most preferably, the ratio is greater than about 99%.

The Tₜ at a given ELP length can be decreased by incorporating a larger fraction of hydrophobic residues at “Xaa” in the ELP sequence. Examples of such suitable hydrophobic Xaa residues include valine, leucine, isoleucine, phenyalanine, tryptophan and methionine. Tyrosine, which is moderately hydrophobic, may also be used. Conversely, the Tₜ can be increased by incorporating residues such as glutamic acid, cysteine, lysine, aspartate, alanine, asparagine, serine, threonine, glycine, arginine, and glutamine; preferably alanine, serine, threonine and glutamic acid.

The Tₜ can also be varied by varying ELP chain length. See, e.g., US Patent App. No. 20010034050 (Chilkoti) and WO9632406 (Urry et al.). The Tₜ generally increases with decreasing ELP domain molecular weight. In low ionic strength buffers, the Tₜ of the lower molecular weight ELPs may be too high for protein purification. A high concentration of NaCl can be used to decrease the Tₜ to a useful temperature. Methods of preparing ELP-encoding nucleotides through genetic engineering techniques are known, such as by recursive directional ligation and concatemerization of a monomer gene.

Linkers may be present in the fusion proteins. Preferred linkers are short, flexible polypeptide domains that allow for the aggregator protein domain or domains to have some conformational flexibility from the intein domain and thereby encourage aggregation by allowing for the necessary physical conformation to be obtained. The linkers are also found within the aggregator protein domain connecting multiple ELP units. In one particular example of a fusion protein, the various domains of the fusion protein are separated by flexible linkers allowing them to function independently. The exception is that very preferably the C-terminus of the intein is joined directly to the N-terminus of the target protein to allow a native target protein to be recovered following intein cleaving. If the C-terminus of the intein is attached to a linker polypeptide that is then attached to the product protein, additional purification steps may be required after intein cleavage to obtain substantially purified product protein. Although linkers may be used, the invention is not limited to fusion proteins containing linkers. For example, the intein can be contiguous with an aggregator protein domain and the product protein domain.

One advantage of the invention is that it can be used with many different types of host cells. For instance, it is envisioned that the purification system can be used with a prokaryotic cell or a eukaryotic cell. Preferably, the host cell is a bacterial cell, a fungal cell, a mammalian cell, an insect cell, a yeast cell, or a plant cell. In one embodiment, the host cell is a bacterial cell, such as E. coli. More particularly, it is envisioned that the invention can be used successfully in mammalian cells.

The plasmid of the invention comprises a nucleotide sequence encoding the fusion protein of the invention. The plasmid can further comprise a promoter sequence, an antibiotic resistance sequence, restriction sites and other elements known in the art that improve the functionality of the plasmid. Preferred is the use of the leaky promoter T7 RNA polymerase such as is described in U.S. Pat. No. 4,952,496.

The invention also relates to a method of purifying a protein comprising isolating the fusion product of the invention from other components of the cell lysate. When the aggregator domain comprises an ELP domain, it may be desirable, after first removing the insoluble cell components, to separate the fusion protein from the soluble portion of the cell lysate by precipitating the fusion protein, removing the soluble cell lysate, and then dissolving the fusion protein. An ELP fusion protein precipitate can usually be dissolved by decreasing the ionic strength of the suspension, decreasing the temperature, or both. Suitable separation methods include centrifugation, filtration such as cross-flow diafiltration and other means known in the art. In a particular embodiment the diafiltration uses nanoporous membranes.

In another aspect the invention comprises using the method in a robotic system to purify protein libraries for screening. The purification system of the present invention can be highly automated and thus is suitable for high through-put screening. Thus, the invention comprises a method of purifying a protein library comprising:
(a) expressing a plurality of polynucleotide sequences in a plurality of host cells, each sequence encoding a fusion protein comprising at least one ELP domain, an intein and a product protein domain,
(b) separately lysing the host cells or allowing the host cells to secrete to form a plurality of first suspensions,
(c) removing insoluble cell components from the plurality of first suspensions to produce a plurality of first protein solutions,
(d) adjusting one or more conditions of temperature, salt content, pH and solvent content to cause self-aggregation of each fusion protein in the plurality of first protein solutions, and
(e) collecting the aggregated fusion proteins. Optionally the method comprises dissolving the fusion proteins. In one embodiment, the fusion proteins comprise the protein library which can be used for screening.

The method of the invention can further comprise
(f) adjusting one or more conditions of temperature, salt content, pH and sulfhydryl content such that the intein self-cleaves from the product protein domain to form an ELP-intein fusion and a product protein, and
(g) separating the product protein from the ELP-intein fusion, wherein the intein is located between the product protein domain and the ELP domain. In an embodiment of the invention, the product proteins comprise the protein library which can be used for screening.

The invention is also directed to a microprocessor-controlled system capable of purifying a protein library according to a method above. The system can comprise one or more microprocessors and an instruction set which controls the operations.

### EXAMPLES

### Example 1

### Purification using ELP as Aggregator-Methods

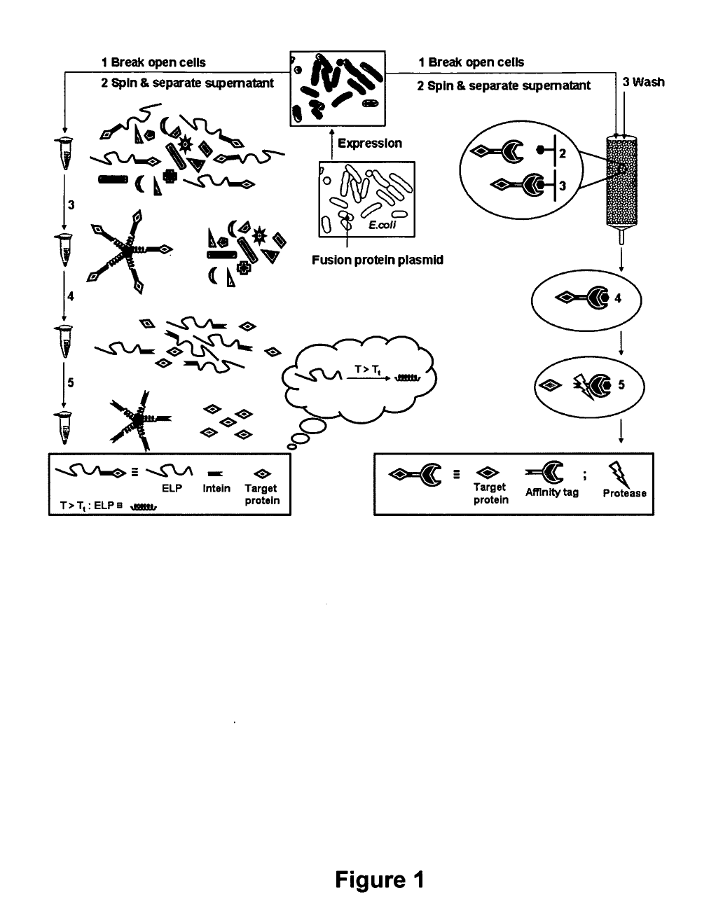

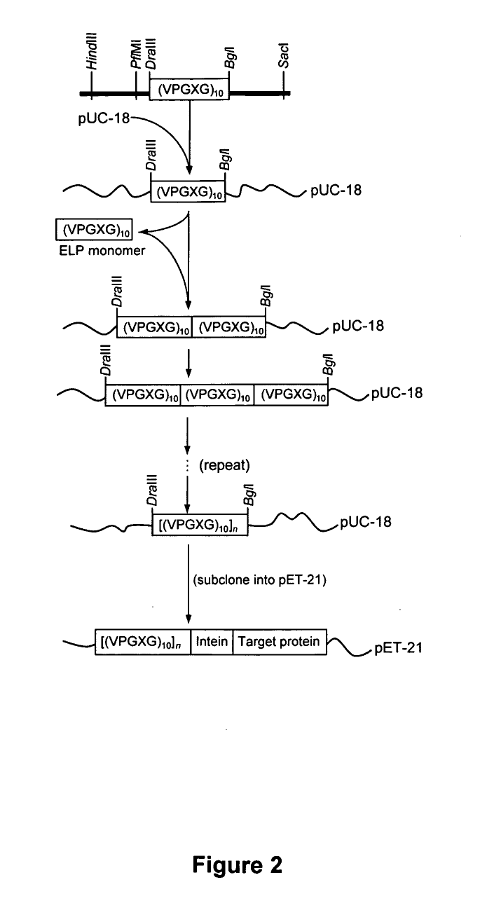

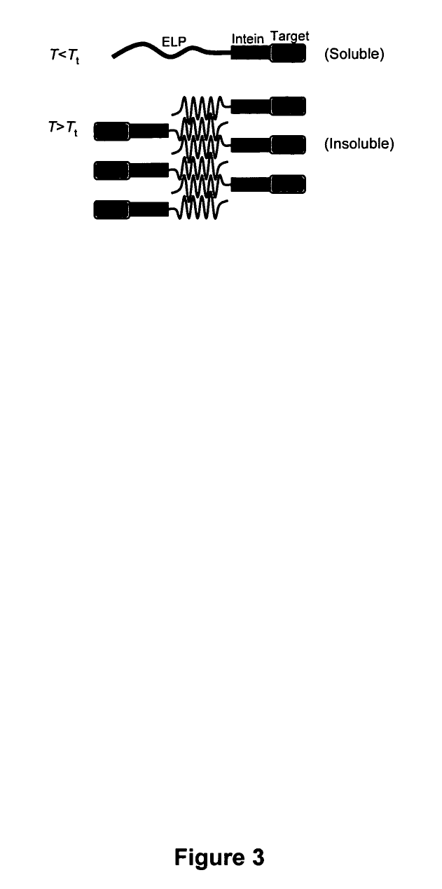

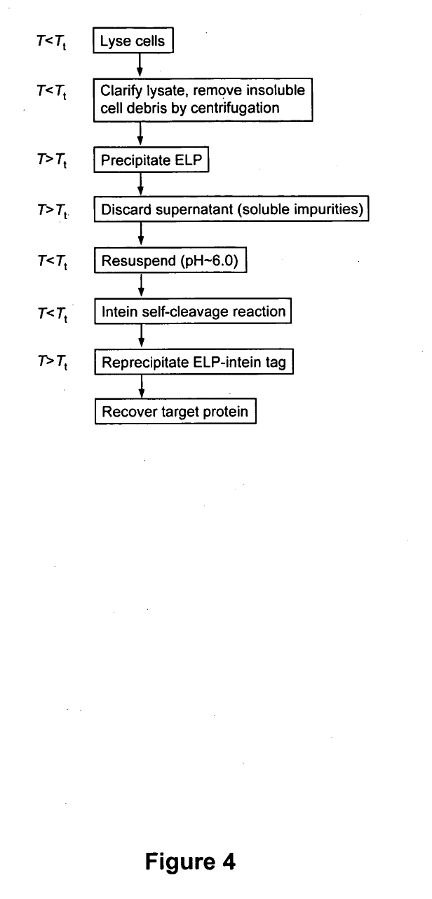

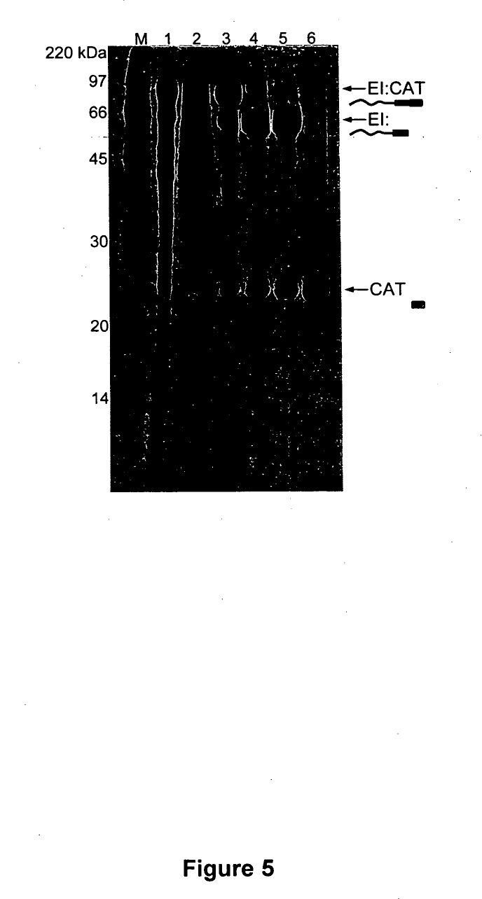

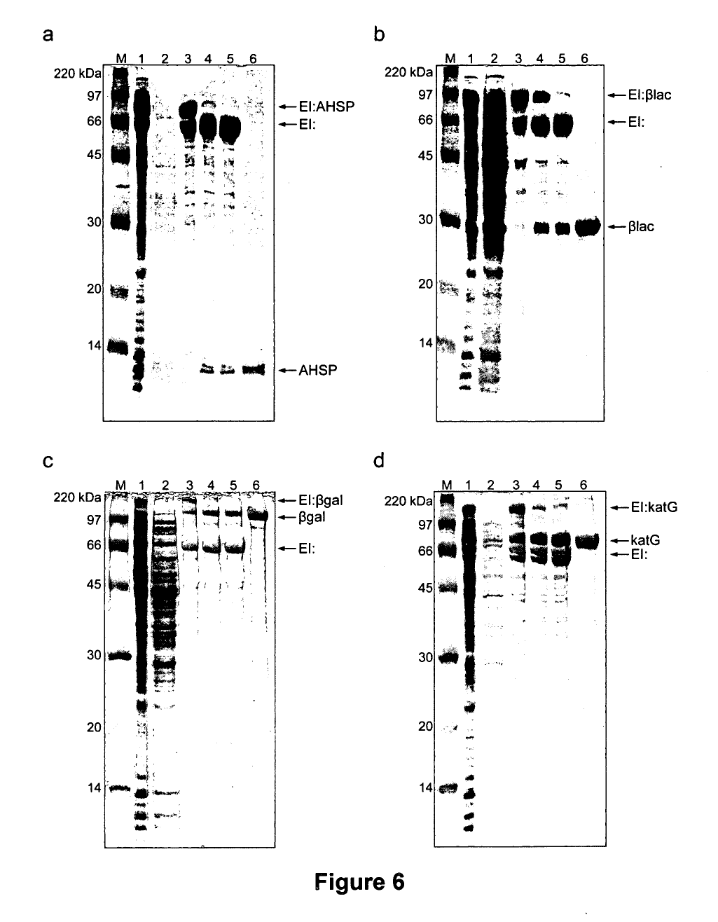

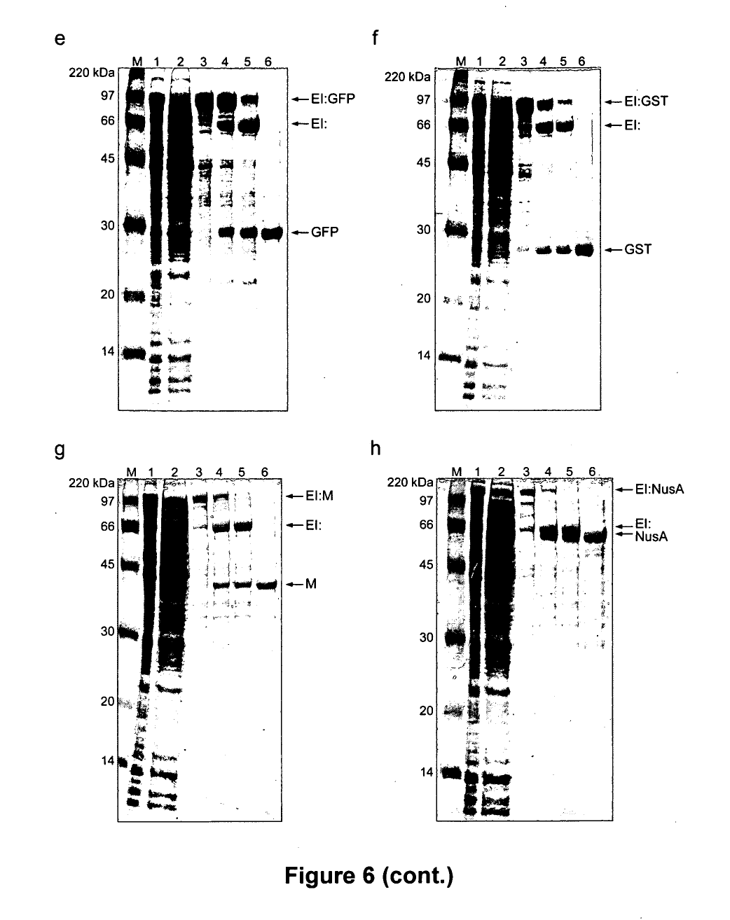

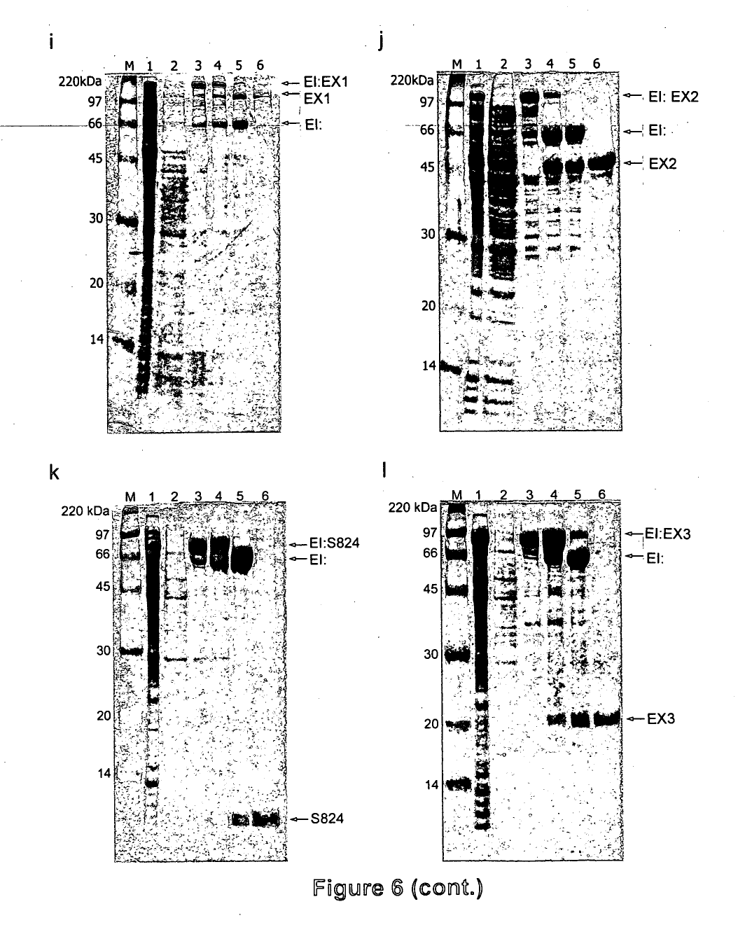

A general scheme for protein purification using ELP-intein-tagged proteins is shown in FIGS. 1-4. In FIG. 2 is illustrated a library of ELP tags of various lengths generated using recursive cloning in pUC-18. The library is subcloned into pET-21 in fusion to an engineered self-cleaving mini-intein and a number of product proteins expressed in E. coli BLR cells. Details of the methods are presented below. In FIG. 3 is shown that the ELP-intein tag is designed to self-associate into an insoluble core upon heating to a temperature above the ELP transition temperature (Tₜ). This process results in precipitation of the fusion protein, which is ELP-intein-product protein, also known as ELP-intein-target protein. The intein and the product protein domains remain properly folded. FIG. 4 illustrates the purification of the fusion protein by a series of simple steps, as described in detail below. Briefly, the cells are lysed and the lysate clarified. Mild warming and centrifugation (or other separation technique) separate the fusion protein from soluble cell debris and, ultimately, the product protein from the self-cleaved ELP tag. FIG. 5 shows the purification of an exemplary protein, CAT. In the SDS-PAGE illustrations of FIGS. 5 and 6, the lanes 1 correspond to samples of the initial ELP precipitate formed after lysing the cells and removing insoluble cell debris as shown in FIG. 4. Lanes 2 correspond to the discarded supernatants. Lanes 3 correspond to an early stage in the intein self-cleavage step. Lanes 5 correspond to a late stage in the intein self-cleavage reaction. Lanes 6 correspond to a sample of the recovered target (product) proteins. For example, lane 3 in FIG. 5 shows that the fusion protein is the predominant material. Lanes 4 and 5 show the kinetics of the cleavage reaction. Lane 6 of FIG. 5 shows the presence of substantially purified CAT.

Cloning, media and expression. The polynucleotide encoding a 10-repeat polypeptide of Val-Pro-Gly-Xaa-Gly (SEQ ID NO:1) was generated by eight overlapping oligonucleotides. Xaa was chosen to be Val:Ala:Gly in a 5:3:2 ratio. These oligos were designed for optimized E. coli expression and formed the building block of (VPGXG)₁₀ which is encoded by SEQ ID NO:3.

The four forward primers are primer FI:

(SEQ ID NO:4)

5′GTTGTTGTTCCATGGGGTTAAGCTTCATATGGGCCACGGCGTGGGT
3′,
primer FII:

(SEQ ID NO:5)

5′TGTTCCGGGTGGCGGTGTGCCGGGCGCAGGTGTTCCTGGTGTAGGTGT
GCCGGG3′,
primer FIII:

(SEQ ID NO:6)

5′GTGTTGGTGTACCAGGTGGCGGTGTTCCGGGTGCAGGCGTTCCGGGT
GGCGGTGT3′,
and
primer FIV:

(SEQ ID NO:7)

5′CCTCTAGGATCCAATAATTTTGTTTAACTTTAAGAAGGAGATATACA
TATGGGCCACGGCGTGGGTGTTCCG3′.
The four backward primers used were: primer BI:

(SEQ ID NO:8)

5′AACGAGCTCACCAGCCCGCCCGGCACACCGCCACCCGGAACGCCT
3′,
primer BII:

(SEQ ID NO:9)

5′GCCACCTGGTACACCAACACCCGGCACACCAACACCCGGCACACCTA
CACCAGG3′,
primer BIII:

(SEQ ID NO:10)

5′GCACACCGCCACCCGGAACACCCACGCCCGGAACACCCACGCCGTGG
CCCATATG3′,
primer BIV:

(SEQ ID NO:11)

5′GTTGGCGAATTCTGAAATCCTTCCCTCGATCCCGAGGTTGTTGTTAT
TGTTATTGTTGTTGTTGTTACTAGTCCCGCCCGGCACACCGCCACC3′.

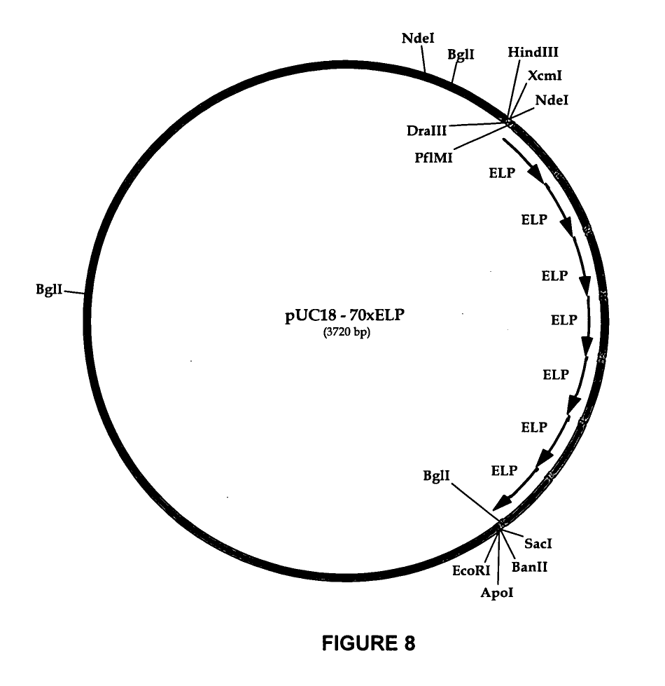

Subcloning of nucleic acids encoding (VPGXG) units was carried out in pUC-18 using the two restriction sites HindIIII and SacI. The first forward primer included a PflMI and DraIII upstream, and the first reverse primer a BglI site downstream flanking the nucleotide sequence corresponding to the (VPGXG)₁₀ sequence. After sequencing, these sites were used for recursive directional ligation to duplicate the nucleic acid encoding the (VPGXG)₁₀ sequence eleven times and generate the polynucleotide encoding the (VPGXG)₁₁₀ sequence (SEQ ID NO:12). Moreover, other nucleic acids encoding (VPGXG)ₙ sequences were prepared, where n=10 (SEQ ID NO:3); n=20 (SEQ ID NO:13), n=30 (SEQ ID NO:14); n=40 (SEQ ID NO:15); n=50 (SEQ ID NO:16); n=60 (SEQ ID NO:17); n=70 (SEQ ID NO:18); n=80 (SEQ ID NO:19); n=110 (SEQ ID NO:12); n=160 (SEQ ID NO:20); n=320 (SEQ ID NO:21); and n=480 (SEQ ID NO:22). In each step the pUC-18:(VPGXG)₁₀ clone was digested with DraIII and BgI to generate the nucleic acid encoding the (VPGXG)₁₀ insert. In addition pUC-18:(VPGXG)₁₀ was also digested once with BglI to generate a single cut vector. Ligation of these two linear pieces generated pUC-18:(VPGXG)₂₀. Subsequent additional (VPGXG)₁₀ units could be added either one-by-one as described above or in multiples by using pUC-18:(VPGXG)ₙ to generate both the insert and the cloning vector. The nucleotide sequence for pUC-ELP₇₀ is shown in SEQ ID NO:23 and a vector map for pUC-18-ELP₇₀ is in FIG. 8. In addition to the above mentioned sites the first forward primer included a BamHI site. The first reverse primer also included an EcoRI site downstream from a spacer sequence of 10 asparagines. The BamHI and EcoRI sites were used to subclone the ELP library into pET-21(+) (Amp^{R}) (Novagen, Madison, Wis.) featuring the T7lac promoter for expression. The sequence of the pET/ELP-I-CAT is found at SEQ ID NO:24. E. coli BLR (DE3) (F⁻ ompT hsdS_{B} (r_{B}⁻m_{B}⁻) gal dcm (DE3) Δ(srl-recA)306::Tn10 (Tet^{R})) (Novagen, Madison, Wis.) was the host strain used for cloning and expression throughout. The engineered Mycobacterium tuberculosis (Mtu) recA mini-intein was PCR amplified from a previous plasmid pMΔI†T-CM and flanked by EcoRI and BsrGI. These sites were used to clone the intein downstream of the ELP gene.

Product protein genes for α-hemoglobin stabilizing protein (AHSP) (SEQ ID NO:25), β-lactamase (β-lac) (SEQ ID NO:26), β-galactosidase (β-gal) (SEQ ID NO:27), catalase (katG) (SEQ ID NO:28), chloramphenicol acetyl transferase (CAT) (SEQ ID NO:29), the green fluorescent protein (GFP) (SEQ ID NO:30), glutathione-S-transferase (GST) (SEQ ID NO:31), the maltose binding protein (M) (SEQ ID NO:32), NusA (SEQ ID NO:33), and S-824 (a completely synthetic protein) (SEQ ID NO:34) were PCR amplified, flanked by BsrGI or AgeI upstream and HindIII or NotI downstream, and inserted downstream from the intein. For AHSP, a flexible linker (SSGLVPRGS) (SEQ ID NO: 35) separated the intein from the AHSP. Orbital shaker baths (300 rpm) and 5 ml culture tubes were used throughout. Overnight cultures were grown in Luria-Bertani medium supplemented with 200 μg/ml ampicillin. Terrific Broth (12 g tryptone, 24 g yeast extract, 0.017M KH₂PO₄, 0.072M K₂HPO₄) supplemented with 200 μg/ml ampicillin was inoculated with the fresh overnight in a 1:100 ratio and the samples were grown at 37° C. for 4 hours or until the OD₆₀₀ reached 0.8. Samples were then transferred to a low temperature shaker bath and grown for another 48 hours between 15-19° C.

Lysis, cleavage and recovery. Cell pellets from a 1 ml sample were first resuspended in 500 μl of lyzozyme buffer (10 mM Tris-HCl pH 8.5, 2 mM EDTA, 1 mg/ml Lyzozyme, trace amounts of DNase and RNase, and 2 mM DTT if necessary (e.g. GST)) and chilled on ice for 45 minutes. Lysis was completed by three sequential freeze-thaw cycles with liquid nitrogen. Samples were spun in a bench-top centrifuge at 4° C. for 5 minutes to pellet insoluble cell debris and the supernatant was transferred to a fresh tube. An equal volume of NaCl (3M) solution was added and mixed with the sample. The resulting sample was heated to 30° C. for 10 minutes followed by centrifugation at 30° C. for 5 minutes. The transition temperature used here is to ensure ELP precipitation. The supernatant from this heat cycle was discarded and the pellet fully resuspended and dissolved in cold (4° C.) pH 6.0 buffer (1× PBS, 40 mM Bis-Tris, pH 6.0 with 2 mM EDTA and 1 mM DTT added if necessary). At this point the sample was incubated at room temperature (18° C.) for the duration of the cleavage reaction. Once the cleavage time course was completed, the heat cycle was repeated. One sample volume of NaCl (3M) was added and mixed with the sample before heating at 30° C. for 10 minutes. At this point the sample was spun for 4 minutes at 30° C. and the supernatants transferred to a fresh tube. The salt content of this purified sample was reduced to 700 mM by adding 2 volumes of the pH 6.0 buffer and concentrating the sample using a Microcon filter devices from Millipore, Bedford, Mass., to its original volume prior to salt addition.

Protein content quantification and activity assays. Protein contents in the final purified samples were measured using the Bradford method using the Bio-Rad Assay Kit (Bio-Rad, Hercules, Calif.). β-galactosidase activity assay was measured in reaction with o-Nitrophenyl β-D-galactopyranoside using the β-gal Activity Assay kit (Stratagene, La Jolla, Calif.). β-lactamase activity was determined spectrophotometrically using nitrocefin hydrolysis (Becton Dickinson, Franklin Lakes, N.J.). Glutathione-S-transferase activity was measured in reaction with 1-chloro-2,4-dinitrobenzene (CDNB) using the GST-Tag Assay Kit (Novagen, Madison, Wis.). Catalase activity was measured using a two-step reaction for decomposition of H₂O₂. The Catalase Assay Kit (CALBIOCHEM, EMD Biosciences, Madison, Wis.) with the chromogenic buffer of 4-aminophenazone, 3,5-dichloro-2-hydroxybenzenesulfoic acid was used to monitor absorbance changes at 520 nm. Affinity of maltose binding protein after purification was verified by adding amylase resin to the sample and centrifugation to verify disappearance of maltose binding protein in the supernatant sample by SDS-PAGE. Recovery and purification fold data for all proteins were based on comparison of precursor fusion activity with that of the purified protein.

Oxidation assay for α-hemoglobin. Purified AHSP at a concentration of 25 μM was incubated with equal molar ratio of α-Hemoglobin (αHb) on ice for 30 min in 300 μl assay buffer (20 mM sodium phosphate buffer at pH 7.4, and 150 mM NaCl). The absorbance spectra from 450-700 nm were recorded every 15 min for 270 min at room temperature. The oxidation rate constant of αHb in the initial stage was calculated according to first-order kinetics. Acceleration of αHb oxidation by AHSP is the ratio of oxidation rate constant between αHb with and without AHSP. Literature value calculation was based on the data in FIG. 5D of the reference.

GFP fluorescence data. The final purified protein fraction volume was 150 μl. From this volume 30 μl of sample from GFP and CAT (control) were mixed with 1 ml of Tris-HCl (10 mM, pH 8.5). Tris-HCl was taken as the background sample. Excitation wavelength was set to 489 nm in an F-4500 FL Spectrophotometer (Hitachi, Ludlow, Ky.). Wavelength emission scans in the range of 450 nm to 580 nm were collected.

Heme binding assay. Purified S-824 samples were assayed by adding 40 μl of sample to 1 ml Hemin supplemented buffer (100 mM sodium phosphate buffer pH 7.0, 30 μM Hemin (Sigma, St. Louis, Mo.). Absorption was then measured spectrophotometrically at 412 nm relative to background and was compared to negative controls.

### Example 2

### Protein Purification Using ELP-Intein Fusion Proteins

The scheme and methods of Example 1 were used to construct a small library of ELPs of varying lengths and insert it into the expression vector pET-21(+) under the control of a T7 promoter. We then used dynamic light scattering to evaluate the ability of each ELP to precipitate efficiently in fusion to the large and highly soluble NusA protein, and determined the approximate Tₜ for each case. The Tₜ of a fusion protein depends, in part, on the properties of all the protein components. Ultimately we chose (VPGXG)₁₁₀ because it fulfilled our design objectives: a moderate transition temperature (below 30° C.) in high salt buffers, efficient precipitation above Tₜ regardless of the fusion context and a short length. We then used a previously reported cloning strategy to insert other protein sequences immediately following the carboxy-terminal His-Asn dipeptide of the intein in the ELP-intein fusion. This strategy retains the conserved C-terminal amino acids necessary for the intein cleavage reaction, and allows the recovery of a native product protein with no additional amino acids.

We purified chloramphenicol acetyl transferase using an ELP-intein-CAT (EI:CAT) fusion and followed the purification by SDS-PAGE analysis (FIG. 5). Growth in Terrific Broth medium at 18° C. for 48 h without induction allowed a buildup of the EI:CAT fusion in E. coli BLR cells. After recovery and gentle disruption in a pH-8.5 buffer at 4° C., we used centrifugation to separate the insoluble cell debris from the soluble lysate components (FIG. 5, lane 1). To reduce the ELP transition temperature, we added NaCl to the cleared lysate to a 1.5 molar final concentration. In subsequent work, we have found that 0.5 to 1.0 molar NaCl is sufficient to reduce the transition temperature. We then warmed the sample to 30° C. for 10 minutes and centrifuged at the same temperature for 5 minutes to pellet the ELP precipitant. SDS-PAGE analysis of the discarded supernatant solution indicated an absence of the EI:CAT fusion, suggesting nearly full recovery of the fusion (FIG. 5, lane 2). We then resuspended the translucent EI:CAT pellet at 4° C., in a low-salt pH-6.0 buffer to solubilize the ELP protein and initiate the self-cleavage reaction. Analysis of the resuspended material indicates near-complete recovery and solubility of the ELP fusion (FIG. 5, compare lanes 1 and 3). Additional tests on this and other proteins indicated that there was no detectable insoluble material remaining after this resolubilization step. For efficient cleavage of the intein, we subsequently incubated the ELP fusion at 18-22° C. Analysis of samples taken at 4 and 25 h indicated that EI:CAT cleaves over time to yield the ELP-intein tag and the cleaved CAT product protein (FIG. 5, lanes 4 and 5). Upon completion of the cleavage reaction, we added NaCl as before and heated the sample to 30° C. for 10 min. The precipitated ELP-intein tag was then separated by centrifugation, and the purified CAT protein was recovered in the supernatant. We then reduced the salt content of the purified product by dilution and ultrafiltration (FIG. 5, lane 6). In addition to CAT, we purified several other proteins of various sizes in a similar manner, and evaluated them for yield, recovery and activity when possible (FIG. 6. See also Table 1).

TABLE 1
Product protein quantification and activity assays.

| | Quantity of | | | | |
|---|---|---|---|---|---|
| | purified | | Specific activity | | |
| Product protein | protein^{a} | Specific activity of | reported in the | Percent | Purification |
| (molecular weight) | (μg/ml) | purified protein | literature | recovery^{b} | fold^{b} |
| β-hemoglobin stabilizing | 104.1 ± 9.1 | Acceleration of rate | Acceleration of rate | NA | NA |
| protein (AHSP) | | of μHb oxidation by | of μHb oxidation by | | |
| (12 kDa) | | AHSP: 6.2 | AHSP: 5.8^{j} | | |
| β-lactamase | 70.3 ± 5.1 | 317.8 units/mg^{c} | 240 units/mg^{k} | 26.5 | 14.7 |
| (29 kDa) | | | | | |
| β-galactosidase | 122.3 ± 10.9 | 358.5 units/mg^{d} | 250-1,200 units/mg^{e} | >100^{f} | 45.0^{f} |
| (116 kDa) | | | | | |
| Catalase | 79.8 ± 7.8 | 703.3 units/mg^{g} | 275-1,486 units/mg | 29.6 | 14.6 |
| (80 kDa) | | | (ref. 14) | | |
| Glutathione S-transferase | 118.0 ± 17.8 | 75.7 units/mg^{h} | 25-125 units/mg^{e} | 18.2 | 12.9 |
| (GST) (26 kDa) | | | | | |
| Green fluorescent protein | 110.2 ± 6.1 | 511 nm fluorescence | 511 nm fluorescence | 21.5 | 10.5 |
| (27 kDa) | | | | | |
| Maltose binding protein | 46.4 ± 4.0 | Binds maltose resin | Binds maltose resin | NA^{l} | NA |
| (41 kDa) | | | | | |
| S-824 (12 kDa) | 45.1 ± 5.2 | Binds heme (412 nm)^{l} | Binds heme (412 nm)^{l} | NA | NA |
| Chloramphenicol acetyl | 96.3 ± 5.2 | ND^{l} | ND | ND | ND |
| transferase (26 kDa) | | | | | |
| NusA (55 kDa) | 56.4 ± 2.7 | NA | NA | NA | NA |

^{a}Yield of protein from a shake-flask cell culture with approximate dry cell weight of 5.65 ± 1.9 mg/ml.
^{b}Purification fold and percent recovery were calculated from the relative activity of the total lysate before purification and the activity of the purified protein.
^{c}One unit of β-lactamase hydrolyzes 1.0 μmole of nitrocefin per minute at 25° C.
^{d}One unit of β-galactosidase hydrolyzes 1.0 μmole of o-nitrophenyl β-D-galactopyranoside (ONPG) to o-nitrophenol and D-galactose per min at 25° C.
^{e}Range of activities of commercially available enzymes available from Sigma-Aldrich.
^{f}Recovery and purification fold were overestimated owing to lower enzyme activity in this fusion.
^{g}One unit of catalase decomposes 1.0 μmole of H₂O₂ in 1 min at pH 7.0 at 25° C.
^{h}One unit of GST conjugates 1.0 μmole of 1-chloro-2,4-dinitrobenzene (CDNB) in 1 min at pH 6.5 at 25° C.
ⁱNA, not applicable; ND, not determined.
^{j}Trabbic-Carlson, et al., Protein Science 13, 3274-3284 (2004).
^{k}Hearn et al., J. Mol. Recognit. 14, 323-369 (2001).
^{l}Claiborne et al., J. Biol. Chem. 254, 4245-4252 (1979).

### Example 3

### Purification of Additional Proteins

Additional product proteins were purified by the method of Example 2 using ELP-intein fusion proteins as the intermediates. The results are shown in FIG. 6, which use the same lane format as discussed for FIG. 5. The product proteins are (a) AHSP, (b) β-lactamase, (c) β-galactosidase, (d) E. coli catalase, (e) green fluorescent protein, (f) glutathione S-transferase, (g) maltose binding protein, (h) Nus A, (i) experimental protein 1 (EX1), (j) experimental protein 2 (EX2), (k) S824 and (l) experimental protein 3 (EX3). The experimental proteins are proteins obtained from collaborators whose structures and functions were not fully known at the time the experiments were performed.

### Example 4

### Full Dissolution of Precipitated ELP Fusion Proteins

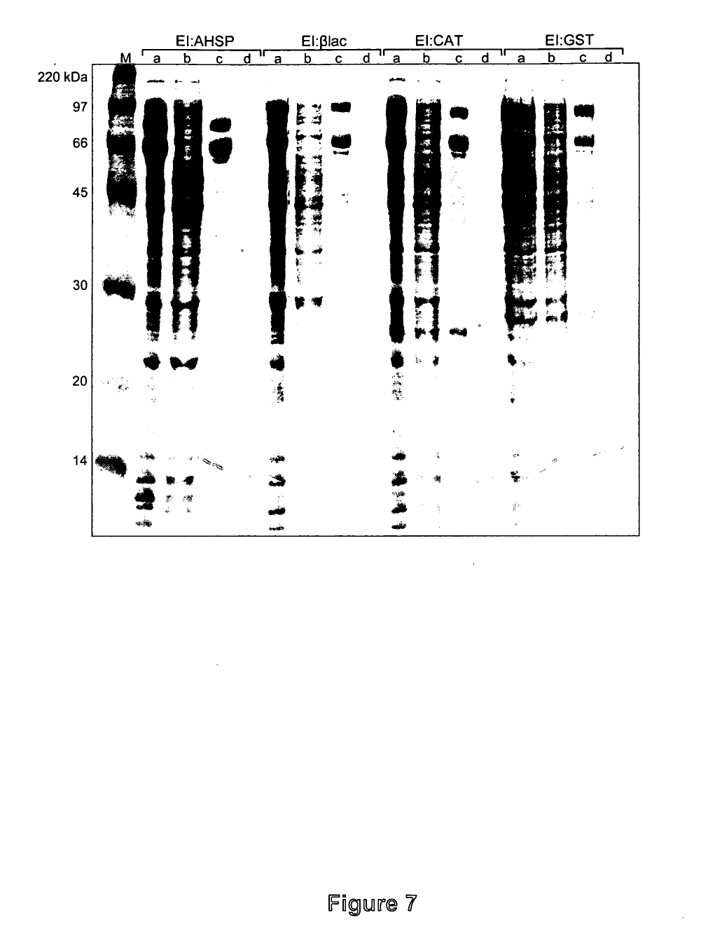

Four proteins were examined for the presence of irreversibly precipitated material following the first thermal cycling step of the purification procedure according to Example 2. Following the low temperature resuspension of the ELP fusion protein at pH 6.0, the solution was re-centrifuged to pellet any remaining insoluble material and the supernatant recovered. The pellet was dissolved in the same volume of SDS gel loading buffer and boiled to effect dissolution. Results are shown in FIG. 7 (lanes a-c). Lane d corresponds to the dissolved material from the inside of the tube after soluble ELP fusion protein was removed. Thus soluble ELP fusion protein is almost fully recovered by dissolution in pH 6.0 buffer at low temperature.

Results are shown for EI: AHSP, EI: β-lac, EI: CAT and EI: GST, as indicated. Table 3 also shows the results of this experiment. The presence of some soluble cleaved product in lanes c for EI: CAT and EI: GST results from pH 6.0—induced cleaving.

Over the course of this work, we tested several conditions for cell growth and protein expression. We noted that the presence of the ELP fusion protein generally slowed growth, and our attempts to induce high levels of expression over a few hours (as is typical when using the T7 promoter) were not effective. Without limiting the invention to any particular theory, we hypothesize that this is due to the large number of amino acid repeats in the ELP, (VPGXG)₁₁₀, although media supplemented with glycine or valine did not change the growth and expression levels noticeably. However, growth at 18° C. with leaky expression from the T7 promoter yielded strongly overexpressed product after 24 h, with maximum expression typically occurring at around 48 h. This result is consistent with previously reported observations by other investigators. For the aforementioned reasons, as well as to suppress premature intein cleavage, we grew cells at 18° C. for 48 h without induction in Terrific Broth as the standard condition for all the samples. We anticipate that expression may be improved in the future by using optimized expression strains and growth conditions to suit specific product proteins.

### Example 5

### Purification of Precipitated ELP Fusion using Crossflow Filtration

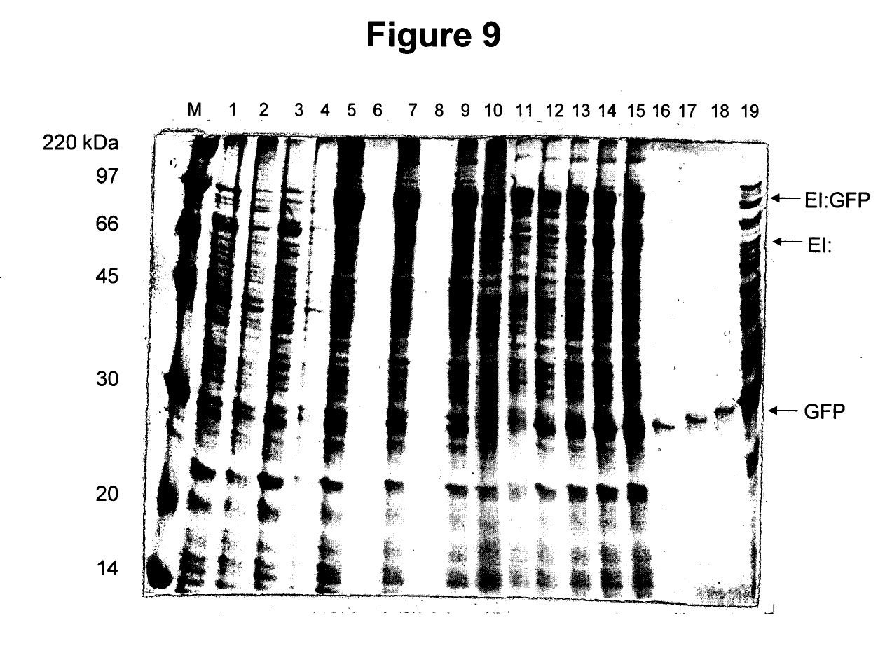

We purified the green fluorescent protein (GFP) using an ELP-intein fusion in combination with crossflow filtration. In this case, crossflow filtration over a nanoporous membrane is used to separate the precipitated ELP-intein-product protein from the soluble cell lysate components, and to subsequently separate the cleaved ELP-intein tag from the GFP product protein. FIG. 9 illustrates the purification of GFP by a series of simple steps, as described in detail below. Briefly, the cells were lysed and the whole-cell lysate was clarified by centrifugation (FIG. 9, lane 1). Salt was then added to the clarified lysate to a final concentration of 1.5 molar, and the solution was heated to 37° C. to precipitate the uncleaved ELP-Intein-GFP precursor. The precipitated ELP fusion was then separated by diafiltration over a 990 kDa ultrafiltration membrane. The separation of the retained precipitant is shown in lanes 3, 5, 7 and 9 of FIG. 9. The filtrate containing the removed soluble cell components are shown in lanes 2, 4, 6, 8 and 10. Back-pulsing of the filter was used to maintain a high flux through the membrane as is common in this type of separation, but is not required for this type of separation. The temperature was then dropped to room temperature (approximately 18° C.) and the cleaving reaction was initiated by addition of acid to shift the buffer pH to 6.5. The progress of the cleaving reaction at 0, 1, 4, 10 and 25 hours is shown in lanes 11, 12, 13, 14 and 15, respectively. The temperature was then raised again to 37° C. to precipitate the cleaved ELP-intein tag, leaving the GFP product protein in solution. Filtration of the resulting precipitate allowed the GFP to be recovered in the filtrate, while the ELP-intein was retained by the membrane. GFP containing filtrate samples are shown in lanes 16, 17 and 18. Lane 19 is a sample of the protein released during membrane cleaning and regeneration.

The simplicity of the membrane filtration process described in this example has allowed up to scale the purification to 1 liter of cell broth while retaining high productivity and yield. This simplicity, combined with the ready availability of commercial filtration equipment at several scales, will allow this process to be used at large scale for inexpensive protein production.

Advantages of the invention. A strength of this purification method is that the conditions over which it is effective are quite broad, thus providing great flexibility in its implementation. The ELP transition temperature can be adjusted using salt concentration whereas the intein cleaving reaction can take place over a wide range of conditions. Some optimization will be required for new, uncharacterized products on a case-by-case basis, as is true of any purification method. One of ordinary skill in the art would, however, be able to apply the methods and techniques of the invention to the expression and purification of any desired product protein, based on the extensive guidance provided herein. The presentation of prototypes here aims to exemplify simple means for protein purification that eliminate the high cost and complexity associated with column operation. Although the reduction in cost is somewhat offset by the long induction time and large tags in the fusion proteins, these issues are minor when taken in the context of conventional protein expression and purification. For example, the additional expression time required for the ELP system represents only a small percentage increase in a typical process time. In most of the cases we have shown, the intein cleaving reaction is essentially complete in 4-10 h, making it competitive with any conventional chromatography process, and the yields we report are reasonable in comparison to those obtained using previously reported ELP strategies and conventional affinity methods. Furthermore, in one aspect the invention comprises the simple mechanical recovery of precipitated fusion protein by tangential-flow microfiltration or continuous centrifugation. This is believed to be favorable to scale the process up for industrial use.

An advantage of the method of the invention is that cell debris is effectively removed from the fusion protein.

All references cited herein are incorporated herein by reference in their entirety.

We claim:

1. A fusion protein comprising:
(a) a product protein domain,
(b) a self-cleaving intein, and
(c) at least one aggregator protein domain capable of self-association and precipitation that comprises one or more elastin-like protein (ELP) domains;
wherein the intein is located between the product protein domain and the aggregator protein domain.

2. The fusion protein of claim 1 wherein the intein is ΔI-CM.

3. The fusion protein of claim 1 wherein the ELP domain comprises the sequence (Val-Pro-Gly-Xaa-Gly)ₙ, wherein n has a value from one to about 480 and Xaa is the same or different and is any natural or synthetic amino acid.

4. The fusion protein of claim 3 wherein n has a value from about ten to about 220.

5. The fusion protein of claim 3 wherein Xaa is selected from Val, Ala, or Gly.

6. The fusion protein of claim 1 in which the at least one aggregator protein domain is covalently attached to the intein by a flexible amino acid linker.

7. A nucleic acid encoding the fusion protein of claim 1.

8. The nucleic acid of claim 7 wherein the product protein domain, the intein, and the aggregator protein domain form a single open reading frame.

9. A plasmid comprising the nucleic acid of claim 7.

10. A cell stably transfected with the nucleic acid of claim 7.

11. The cell of claim 10 wherein said cell is a strain from E. coli.

12. A method of purifying a product protein from a recombinant cell culture comprising:
(a) expressing a nucleotide encoding the fusion protein of claim 1 in a host cell;
(b) allowing the fusion protein to leave the host cell by cell secretion or as a result of cell lysis;
(c) removing insoluble cell culture components from the cell culture to form a first suspension containing the fusion protein;
(d) adjusting one or more of the conditions of temperature, salt content, pH and solvent content of the first suspension to cause self-aggregation and precipitation of the fusion protein forming a first precipitate;
(e) separating the unprecipitated components from the first precipitate;
(f) adding water or solvent to the first precipitate and adjusting one or more of the conditions of temperature, salt content, pH and solvent content such that the fusion protein is resuspended to form a second suspension;
(g) adjusting one or more of the conditions of temperature, salt content, pH and sulfhydryl level of the second suspension such that the intein self-cleaves from the product protein to form an ELP-intein fusion and a separated product protein;
(h) adjusting one or more conditions of temperature, salt content, pH and solvent content such that the ELP-intein fusion self-aggregates and precipitates while the separated product protein remains in solution; and
(i) separating the solution of separated product protein from the ELP-fusion precipitate to yield a substantially purified product protein.

13. The method of claim 12 wherein the first precipitate is formed by warming the first suspension to at or above the transition temperature of the fusion protein.

14. The method of claim 12 wherein the first precipitate is formed by adding salt and warming the first suspension to at or above the transition temperature of the fusion protein.

15. The method of claim 12 wherein the insoluble cell culture components are removed from the cell culture medium by centrifugation, filtration, flocculation or by settling.

16. The method of claim 12 wherein the first precipitate is separated from the unprecipitated components by centrifugation, filtration, flocculation or by settling.

17. The method of claim 12 wherein the ELP domain comprises at least ten repeating units of SEQ ID NO:1.

18. The method of claim 12 wherein the ELP domain has a molecular weight of between 10,000 to 100,000 Daltons.

19. The method of claim 12 wherein the intein is ΔI-CM.

20. The method of claim 12 wherein the temperature of the second suspension is adjusted to 18-22° C. and the suspension is incubated such that the intein self-cleaves from the product protein.

21. A method of purifying a protein library comprising:
(a) expressing a plurality of polynucleotide sequences in a plurality of host cells, each sequence encoding a fusion protein comprising at least one ELP domain, an intein and a product protein domain,
(b) separately lysing the host cells or allowing the host cells to secrete to form a plurality of first suspensions,
(c) removing insoluble cell components from the plurality of first suspensions to produce a plurality of first protein solutions,
(d) adjusting one or more conditions of temperature, salt content, pH and solvent content to cause self-aggregation of each fusion protein in the plurality of first protein solutions, and
(e) separately collecting the aggregated fusion proteins.

22. The method of claim 21 further comprising:
(f) adjusting one or more conditions of temperature, salt content, pH and sulfhydryl content of said aggregated fusion proteins such that an intein self-cleaves from a product protein domain to form an ELP-intein fusion and a product protein, and
(g) separating the product protein from the ELP-intein fusion,
wherein the intein is located between the product protein domain and the ELP domain.

23. A microprocessor-controlled system capable of purifying a protein library according to claim 21.

24. The method of claim 12 further comprising further purifying the product protein.


SEQUENCE LISTING (35 sequences)

SEQ ID NO: 1 - protein, 5 aa, Artificial Sequence
  MISC_FEATURE (4)..(4): “Xaa” can be any natural or synthetic amino acid
VPGXG

SEQ ID NO: 2 - DNA, 504 bp, Artificial Sequence
  Delta-I-CM Intein
gccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaac

SEQ ID NO: 3 - DNA, 196 bp, Artificial Sequence
  Nucleotide Sequence Encoding a 10-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 4 - DNA, 46 bp, Artificial Sequence
  Forward Primer FI
gttgttgttccatggggttaagcttcatatgggccacggcgtgggt

SEQ ID NO: 5 - DNA, 54 bp, Artificial Sequence
  Forward Primer FII
tgttccgggtggcggtgtgccgggcgcaggtgttcctggtgtaggtgtgccggg

SEQ ID NO: 6 - DNA, 55 bp, Artificial Sequence
  Forward Primer FIII
gtgttggtgtaccaggtggcggtgttccgggtgcaggcgttccgggtggcggtgt

SEQ ID NO: 7 - DNA, 72 bp, Artificial Sequence
  Forward Primer FIV
cctctaggatccaataattttgtttaactttaagaaggagatatacatatgggccacggcgtgggtgttccg

SEQ ID NO: 8 - DNA, 45 bp, Artificial Sequence
  Backward Primer FI
aacgagctcaccagcccgcccggcacaccgccacccggaacgcct

SEQ ID NO: 9 - DNA, 54 bp, Artificial Sequence
  Backward Primer FII
gccacctggtacaccaacacccggcacaccaacacccggcacacctacaccagg

SEQ ID NO: 10 - DNA, 55 bp, Artificial Sequence
  Backward Primer FIII
gcacaccgccacccggaacacccacgcccggaacacccacgccgtggcccatatg

SEQ ID NO: 11 - DNA, 93 bp, Artificial Sequence
  Backward Primer FIV
gttggcgaattctgaaatccttccctcgatcccgaggttgttgttattgttattgttgttgttgttactagtcccgcccggcacaccgccacc

SEQ ID NO: 12 - DNA, 1696 bp, Artificial Sequence
  Nucleotide Sequence Encoding an 110-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 13 - DNA, 346 bp, Artificial Sequence
  Nucleotide Sequence Encoding a 20-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 14 - DNA, 496 bp, Artificial Sequence
  Nucleotide Sequence Encoding a 30-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 15 - DNA, 646 bp, Artificial Sequence
  Nucleotide Sequence Encoding a 40-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 16 - DNA, 796 bp, Artificial Sequence
  Nucleotide Sequence Encoding a 50-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 17 - DNA, 946 bp, Artificial Sequence
  Nucleotide Sequence Encoding a 60-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 18 - DNA, 1096 bp, Artificial Sequence
  Nucleotide Sequence Encoding a 70-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 19 - DNA, 1246 bp, Artificial Sequence
  Nucleotide Sequence Encoding an 80-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 20 - DNA, 2446 bp, Artificial Sequence
  Nucleotide Sequence Encoding a 160-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 21 - DNA, 4846 bp, Artificial Sequence
  Nucleotide Sequence Encoding a 320-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 22 - DNA, 7246 bp, Artificial Sequence
  Nucleotide Sequence Encoding a 480-repeat ELP Polypeptide
gtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattc

SEQ ID NO: 23 - DNA, 3720 bp, Artificial Sequence
  pUC-ELP70
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 8292 bp, Artificial Sequence
  pET/ELP-I-CAT
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctaggatccaataattttgtttaactttaagaaggagatatacatatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggtgagctcgaacaacaacaacaataacaataacaacaacctcgggatcgagggaaggatttcagaattcgccctcgcagagggcactcggatcttcgatccggtcaccggtacaacgcatcgcatcgaggatgttgtcggtgggcgcaagcctattcatgtcgtggctgctgccaaggacggaacgctgcatgcgcggcccgtggtgtcctggttcgaccagggaacgcgggatgtgatcgggttgcggatcgccggtggcgccatcctgtgggcgacacccgatcacaaggtgctgacagagtacggctggcgtgccgccggggaactccgcaagggagacagggtggcgcaaccgcgacgcttcgatggattcggtgacagtgcgccgattccggcgcgcgtgcaggcgctcgcggatgccctggatgacaaattcctgcacgacatgctggcggaagaactccgctattccgtgatccgagaagtgctgccaacgcggcgggcacgaacgttcggcctcgaggtcgaggaactgcacaccctcgtcgccgaaggggttgttgtacacaacatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatgagcaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 25 - DNA, 309 bp, Homo sapiens
atggctcttcttaaggccaataaggatctcatttccgcaggattgaaggagttcagcgttctgctgaatcagcaggtcttcaatgatcctctcgtctctgaagaagacatggtgactgtggtggaggactggatgaacttctacatcaactattacaggcagcaggtgacaggggagccccaagagcgagacaaggctctgcaggagcttcggcaagagctgaacactctggccaaccctttcctggccaagtacagggacttcctgaagtctcatgagctcccgagtcacccaccgccctcctcctag

SEQ ID NO: 26 - DNA, 795 bp, Unknown
  beta-Lactamase
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 27 - DNA, 3075 bp, Escherichia coli
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 28 - DNA, 2181 bp, Escherichia coli
atgagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtgccaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgcggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaa

SEQ ID NO: 29 - DNA, 660 bp, Unknown
  Chloramphenicol Acetyl Transferase
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 30 - DNA, 717 bp, Unknown
  Green Fluorescent Protein
atgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 31 - DNA, 663 bp, Unknown
  Glutathione S-Transferase
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggattaa

SEQ ID NO: 32 - DNA, 1116 bp, Unknown
  Maltose Binding Protein
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgtga

SEQ ID NO: 33 - DNA, 1488 bp, Unknown
  NusA
atgaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggacatcgccgttgaagccggtaatctggcgcaggcgattggccgtaacggtcagaacgtgcgtctggcttcgcaactgagcggttgggaactcaacgtgatgaccgttgacgacctgcaagctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaatatttgctggttcggtgacgaagcgtga

SEQ ID NO: 34 - DNA, 309 bp, Unknown
  S-824
atgtatggcaagttgaacgacctgctggaagacttgcaagaggtgctgaagaacctccacaaaaactggcacggtggcaaagacaacctgcacgacgtcgacaaccacttgcagaacgtcatcgaagacatccacgacttcatgcaaggcggtggcagcggcggcaagctgcaagagatgatgaaagagttccaacaggtgttggacgaactcaacaaccacttgcaaggcggtaaacacaccgtgcaccacatcgaacaaaacatcaaagagatcttccaccacttggaagagctcgtccaccgttaa

SEQ ID NO: 35 - protein, 9 aa, Artificial Sequence
  Flexible Linker
SSGLVPRGS